(19) United States
(12) Patent Application Publication
(10) Pub. No.: US 2019/0292305 A1
(43) Pub. Date: Sep. 26, 2019

(54) URETDIONE BASED POLYURETHANE COMPOSITIONS

(71) Applicants: Covestro LLC, Pittsburgh, PA (US); Covestro Deutschland AG, Leverkusen (DE)

(72) Inventors: Alan Ekin, Coraopolis, PA (US); David P. Zielinski, Cranberry Township, PA (US); Dorota Greszta-Franz, Solingen (DE); Florian Johannes Stempfle, Koln (DE)

(73) Assignees: Covestro LLC; Covestro Deutschland AG

(21) Appl. No.: 15/933,487

(22) Filed: Mar. 23, 2018

(51) Int. Cl. C08G 18/78; C08G 18/79; C08L 75/04

(52) U.S. Cl. CPC C08G 18/7831; C08G 18/7862; C07C 249/02; C08L 75/04; C08G 18/798

(57) ABSTRACT

The present invention provides a composition comprising a polyuretdione resin; and a blocked amine; and optionally, an additive package (e.g. flow control, wetting agent) and a solvent. The present invention further provides a method of forming a biuret comprising the steps of: unblocking a blocked amine in the presence of moisture; and crosslinking the unblocked amine with a polyuretdione resin. The compositions of the present invention are particularly applicable in or as coatings, adhesives, castings, composites, and sealants with good performance and extended pot-life.

### FIELD OF THE INVENTION

The present invention relates, in general to polymers, and more specifically, to uretdione based polyurethane compositions useful as coatings, adhesives, castings, composites, and sealants which are made with polyuretdiones and blocked amines to extend pot-life of the resultant coatings, adhesives, castings, composites, and sealants without diminishing performance.

### BACKGROUND OF THE INVENTION

Polyurethane-forming compositions are widely used in a variety of commercial, industrial and household applications, such as in automotive clear-coat and seat cushion applications. Polyurethane systems that employ isocyanates which are pre-reacted with monofunctional reagents to form relatively thermally labile compounds are called blocked isocyanates. Uretdiones are a type of blocked isocyanate. Uretdiones are typically prepared by dimerizing an isocyanate to form a uretdione with unreacted isocyanate groups which can then be extended with a polyol to form a polymeric material containing two or more uretdione groups in the polymer chain. In some literature, uretdiones are referred to as “1,3-diaza-2,4-cyclobutanones”, “1,3-diazatidin-2,4-diones”, “2,4-dioxo-1,3-diazetidines”, “urethdiones” or “uretidiones”. Typically, the polymer has few, if any, free isocyanate groups, which is achieved by controlling the stoichiometry of the polyisocyanate, polyol and by the use of a blocking agent.

Polyuretdiones and amines react in a very fast fashion and thus do not form stable systems in terms of pot-life. To the best of the present inventors' knowledge, no one has developed a cross-linking approach using blocked amines in combination with uretdiones.

To reduce or eliminate pot-life problems, a need exists in the art for an alternative cross-linking approach to obtain compositions having physical properties similar to those of polyurethane compositions.

### SUMMARY OF THE INVENTION

Accordingly, the present invention attempts to alleviate problems inherent in the art by providing such an alternative cross-linking approach to obtain compositions having physical properties similar to those of polyurethane compositions. Various embodiments of the inventive approach involve crosslinking polyuretdione resins with blocked amines (after unblocking in the presence of moisture) to form biurets. Blocked amines such as aldimines and ketimines will not react with uretdiones until unblocked via moisture (typically obtained from the atmosphere). The presence of aldimines and ketimines in such compositions can extend the pot-life of resultant coatings, adhesives, castings, composites, and sealants.

It is understood that the invention disclosed and described in this specification is not limited to the embodiments summarized in this Summary.

These and other advantages and benefits of the present invention will be apparent from the Detailed Description of the Invention herein below.

### DETAILED DESCRIPTION OF THE INVENTION

The present invention will now be described for purposes of illustration and not limitation. Except in the operating examples, or where otherwise indicated, all numbers expressing quantities, percentages, and so forth in the specification are to be understood as being modified in all instances by the term “about.”

Any numerical range recited in this specification is intended to include all sub-ranges of the same numerical precision subsumed within the recited range. For example, a range of “1.0 to 10.0” is intended to include all sub-ranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited in this specification is intended to include all lower numerical limitations subsumed therein and any minimum numerical limitation recited in this specification is intended to include all higher numerical limitations subsumed therein. Accordingly, Applicants reserve the right to amend this specification, including the claims, to expressly recite any sub-range subsumed within the ranges expressly recited herein. All such ranges are intended to be inherently described in this specification such that amending to expressly recite any such sub-ranges would comply with the requirements of 35 U.S.C. § 112(a), and 35 U.S.C. § 132(a).

Any patent, publication, or other disclosure material identified herein is incorporated by reference into this specification in its entirety unless otherwise indicated, but only to the extent that the incorporated material does not conflict with existing definitions, statements, or other disclosure material expressly set forth in this specification. As such, and to the extent necessary, the express disclosure as set forth in this specification supersedes any conflicting material incorporated by reference herein. Any material, or portion thereof, that is said to be incorporated by reference into this specification, but which conflicts with existing definitions, statements, or other disclosure material set forth herein, is only incorporated to the extent that no conflict arises between that incorporated material and the existing disclosure material. Applicants reserve the right to amend this specification to expressly recite any subject matter, or portion thereof, incorporated by reference herein.

Reference throughout this specification to “various non-limiting embodiments,” “certain embodiments,” or the like, means that a particular feature or characteristic may be included in an embodiment. Thus, use of the phrase “in various non-limiting embodiments,” “in certain embodiments,” or the like, in this specification does not necessarily refer to a common embodiment, and may refer to different embodiments. Further, the particular features or characteristics may be combined in any suitable manner in one or more embodiments. Thus, the particular features or characteristics illustrated or described in connection with various or certain embodiments may be combined, in whole or in part, with the features or characteristics of one or more other embodiments without limitation. Such modifications and variations are intended to be included within the scope of the present specification.

The grammatical articles “a”, “an”, and “the”, as used herein, are intended to include “at least one” or “one or more”, unless otherwise indicated, even if “at least one” or “one or more” is used in certain instances. Thus, the articles are used in this specification to refer to one or more than one (i.e., to “at least one”) of the grammatical objects of the article. By way of example, and without limitation, “a component” means one or more components, and thus, possibly, more than one component is contemplated and may be employed or used in an implementation of the described embodiments. Further, the use of a singular noun includes the plural, and the use of a plural noun includes the singular, unless the context of the usage requires otherwise.

Although compositions and methods are described in terms of “comprising” various components or steps, the compositions and methods can also “consist essentially of” or “consist of” the various components or steps.

In various embodiments, the present invention provides a composition comprising a polyuretdione resin; a blocked amine; and optionally, an additive package (e.g. flow control, wetting agent) and a solvent. In certain embodiments, the present invention further provides a method of forming a biuret comprising the steps of: unblocking a blocked amine in the presence of moisture; and crosslinking the unblocked amine with a polyuretdione resin.

The inventive biuret polymer is particularly applicable in making coatings, adhesives, castings, composites, and sealants.

As used herein, the term “polymer” encompasses prepolymers, oligomers and both homopolymers and copolymers; the prefix “poly” in this context referring to two or more. As used herein, the term “molecular weight”, when used in reference to a polymer, refers to the number average molecular weight, unless otherwise specified.

As used herein, the term “coating composition” refers to a mixture of chemical components that will cure and form a coating when applied to a substrate.

The terms “adhesive” or “adhesive compound”, refer to any substance that can adhere or bond two items together. Implicit in the definition of an “adhesive composition” or “adhesive formulation” is the concept that the composition or formulation is a combination or mixture of more than one species, component or compound, which can include adhesive monomers, oligomers, and polymers along with other materials.

A “sealant composition” refers to a composition which may be applied to one or more surfaces to form a protective barrier, for example to prevent ingress or egress of solid, liquid or gaseous material or alternatively to allow selective permeability through the barrier to gas and liquid. In particular, it may provide a seal between surfaces.

A “casting composition” refers to a mixture of liquid chemical components which is usually poured into a mold containing a hollow cavity of the desired shape, and then allowed to solidify.

A “composite” refers to a material made from two or more polymers, optionally containing other kinds of materials. A composite has different properties from those of the individual polymers/materials which make it up.

“Cured,” “cured composition” or “cured compound” refers to components and mixtures obtained from reactive curable original compound(s) or mixture(s) thereof which have undergone a chemical and/or physical changes such that the original compound(s) or mixture(s) is(are) transformed into a solid, substantially non-flowing material. A typical curing process may involve crosslinking.

The term “curable” means that an original compound(s) or composition material(s) can be transformed into a solid, substantially non-flowing material by means of chemical reaction, crosslinking, radiation crosslinking, or the like. Thus, compositions of the invention are curable, but unless otherwise specified, the original compound(s) or composition material(s) is(are) not cured.

The compositions useful in the present invention comprise a polyisocyanate. As used herein, the term “polyisocyanate” refers to compounds comprising at least two unreacted isocyanate groups, such as three or more unreacted isocyanate groups. The polyisocyanate may comprise diisocyanates such as linear aliphatic polyisocyanates, aromatic polyisocyanates, cycloaliphatic polyisocyanates and aralkyl polyisocyanates.

Particularly preferred in the present invention are those blocked isocyanates known as uretdiones. The uretdiones useful in the invention may be obtained by catalytic dimerization of polyisocyanates by methods which are known to those skilled in the art. Examples of dimerization catalysts include, but are not limited to, trialkylphosphines, aminophosphines and aminopyradines such as dimethylaminopyridines, and tris(dimethylamino)phosphine, as well as any other dimerization catalyst. The result of the dimerization reaction depends, in a manner known to the skilled person, on the catalyst used, on the process conditions and on the polyisocyanates employed. In particular, it is possible for products to be formed which contain on average more than one uretdione group per molecule, the number of uretdione groups being subject to a distribution. The (poly)uretdiones may optionally contain isocyanurate, biuret, allophanate, and iminooxadiazine dione groups in addition to the uretdione groups.

The uretdiones are NCO-functional compounds and may be subjected to a further reaction, for example, blocking of the free NCO groups or further reaction of NCO groups with NCO-reactive compounds having a functionality of 2 or more to extend the uretdiones to form polyuretdione prepolymers. This gives compounds containing uretdione groups and of higher molecular weight, which, depending on the chosen proportions, may also contain NCO groups, be free of NCO groups or may contain isocyanate groups that are blocked.

Suitable blocking agents include, but are not limited to, alcohols, lactams, oximes, malonates, alkyl acetoacetates, triazoles, phenols, imidazoles, pyrazoles and amines, such as butanone oxime, diisopropylamine, 1,2,4-triazole, dimethyl-1,2,4-triazole, imidazole, diethyl malonate, ethyl acetoacetate, acetone oxime, 3,5-dimethylpyrazole, caprolactam, N-tert-butylbenzylamine and cyclopentanone including mixtures of these blocking agents.

Examples of NCO-reactive compounds with a functionality of two or more include polyols. In some embodiments, the NCO-reactive compounds are used in amounts sufficient to react with all free NCO groups in the uretdione. By “free NCO groups” it is meant all NCO groups not present as part of the uretdione, isocyanurate, biuret, allophanate and iminooxadiazine dione groups.

The resulting polyuretdione contains at least 2, such as from 2 to 10 uretdione groups. More preferably, the polyuretdione contains from 5% to 45% uretdione, 10% to 55% urethane, and less than 2% isocyanate groups. The percentages are by weight based on total weight of resin containing uretdione, urethane, and isocyanate.

Suitable polyisocyanates for producing the uretdiones useful in embodiments of the invention include, organic diisocyanates represented by the formula

R(NCO)₂

wherein R represents an organic group obtained by removing the isocyanate groups from an organic diisocyanate having (cyclo)aliphatically bound isocyanate groups and a molecular weight of 112 to 1000, preferably 140 to 400. Preferred diisocyanates for the invention are those represented by the formula wherein R represents a divalent aliphatic hydrocarbon group having from 4 to 18 carbon atoms, a divalent cycloaliphatic hydrocarbon group having from 5 to 15 carbon atoms, or a divalent araliphatic hydrocarbon group having from 7 to 15 carbon atoms. Examples of the organic diisocyanates which are particularly suitable for the present invention include 1,4-tetramethylene diisocyanate, 1,6-hexamethylene diisocyanate (HDI), 2,2,4-trimethyl-1,6-hexamethylene diisocyanate, 1,12-dodecamethylene diisocyanate, cyclohexane-1,3- and 1,4-diisocyanate, 1-isocyanato-2-isocyanato-methyl cyclopentane, 1-isocyanato-3-isocyanatomethyl-3,5,5-trimethyl cyclohexane (isophorone diisocyanate or IPDI), bis-(4-isocyanatocyclohexyl)methane, 1,3- and 1,4-bis(isocyanatomethyl)-cyclohexane, bis-(4-isocyanato-3-methyl-cyclohexyl)-methane, α,α,α′,α′-tetramethyl-1,3- and 1,4-xylene diisocyanate, 1-isocyanato-1-methyl-4(3)-isocyanato-methyl cyclohexane, and 2,4- and 2,6-hexahydrotoluene diisocyanate, toluene diisocyanate (TDI), diphenylmethane diisocyanate (MDI), pentane diisocyanate (PDI)-bio-based, and, isomers of any of these; or combinations of any of these. Mixtures of diisocyanates may also be used. Preferred diisocyanates include 1,6-hexamethylene diisocyanate, isophorone diisocyanate, and bis(4-isocyanatocyclohexyl)-methane because they are readily available and yield relatively low viscosity polyuretdione polyurethane oligomers.

In some embodiments, the uretdiones may comprise from 25% to 95% resin solids in the composition of present invention, excluding solvents, additives or pigments. In other embodiments, from 50% to 95% and in still other embodiments, 80% to 95%. The uretdiones may comprise any resin solids amount ranging between any combinations of these values, inclusive of the recited values. In certain embodiments, the uretdione may contain water to speed the reaction with the amine. In those embodiments, the amount of water is from 0.1% to 5% by weight.

As those skilled in the art are aware, polyuretdiones and amines react in a very rapid fashion and do not form stable systems in terms of pot-life. The present inventors theorized that combining blocked amines such as aldimines, ketimines, or oxazolidines with uretdiones would produce a composition having an extended pot-life. After the blocked amines (e.g., aldimines, ketimines, oxazolidines, enamines, and imidazolidines) are converted back to their respective amines by exposure to moisture, they react with the uretdione immediately and form biurets.

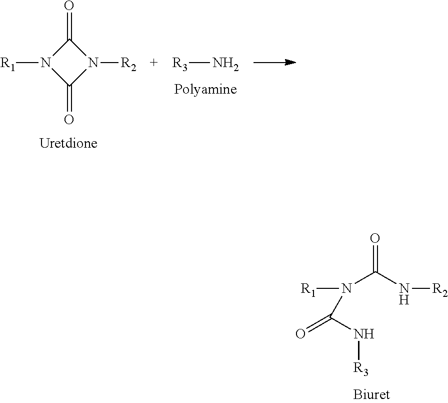

Blocked amines suitable in the present invention include aldimines, ketimines, and oxazolidines. As used herein, the term “aldimine” means an imine that is an analog of an aldehyde. As such, aldimines have the general formula R—CH═N—R′. Aldimines useful in the present invention may be prepared from aldehydes such as acetaldehyde, formaldehyde, propionaldehyde, isobutyraldehyde, n-butyraldehyde, heptaldehyde and cyclohexyl aldehydes, and aldehydes, such as acetaldehyde, formaldehyde, propionaldehyde, isobutyraldehyde, n-butyraldehyde, heptaldehyde and cyclohexyl aldehyde by reaction with an amine. Such amines include, but are not limited to, ethylene diamine, ethylene triamine, propylene diamine, tetramethylene diamine, 1,6-hexamethylene diamine, bis-(6-aminohexyl)ether, tricyclodecane diamine, N,N′-dimethyldiethyltriamine, cyclohexyl-1,2,4-triamine, cyclohexyl-1,2,4,5-tetraamine, 3,4,5-triaminopyran, 3,4-diaminofuran, cycloaliphatic diamines, triaminononane and Jeffamines (polyoxyalkyleneamines, polyethers which contain primary amine groups at the end of the polyether backbone).

As used herein, the term “ketimine” means an imine derived from a ketone having a general formula R₂C═NR. Ketimines useful in the present invention are prepared by the reaction of ketones with amines. Ketones, which may be used to form the ketimine, include, but are not limited to, acetone, methyl ethyl ketone, methyl isopropyl ketone, methyl isobutyl ketone, diethyl ketone, benzyl methylketone, diisopropyl ketone, cyclopentanone, and cyclohexanone. Amines which may be used to form the ketimine include, but are not limited to, ethylene diamine, ethylene triamine, propylene diamine, tetramethylene diamine, 1,6-hexamethylene diamine, bis-(6-aminohexyl)ether, tricyclodecane diamine, N,N′-dimethyldiethyltriamine, cyclohexyl-1,2,4-triamine, cyclohexyl-1,2,4,5-tetraamine, 3,4,5-triaminopyran, 3,4-diaminofuran, cycloaliphatic diamines, triaminononane and Jeffamines (polyoxyalkyleneamines, polyethers which contain primary amine groups at the end of the polyether backbone).

As used herein, the term “oxazolidine” means a five-membered ring compound consisting of three carbon atoms, a nitrogen atom, and an oxygen atom. The oxygen and NH group are the 1 and 3 positions of the ring, respectively, as shown below.

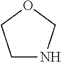

Suitable oxazolidines include, but are not limited to, 4-propyl oxazolidine, 5-methyl oxazolidine, 2,2-dimethyl oxazolidine, 1-butyl-2,2-dimethyl oxazolidine, 1-methyl-2-butyl oxazolidine, 1-ethyl oxazolidine, 1-ethyl-2,2-dimethyl oxazolidine, 1-ethyl-2-isopropyl oxazolidine, carbonato-bis-N-ethyl-2-isopropyl-1,3-oxazolidine, commercially available as INCOZOL LV, 2-(3-heptyl)-N-butyl-1,3-oxazolane, commercially available as INCOZOL 2, and urethane bis-oxazolidines.

In some embodiments, the blocked amines may comprise from 5% to 75% resin solids in the composition of present invention, excluding solvents, additives and pigments. In other embodiments, the blocked amines may comprise from 5% to 50% and in still other embodiments, 5% to 20%. The blocked amines may comprise any resin solids amount ranging between any combinations of these values, inclusive of the recited values.

Examples of suitable solvents are aliphatic and aromatic hydrocarbons such as toluene, xylene, isooctane, acetone, butanone, methyl ethyl ketone, methyl amyl ketone, methyl isobutyl ketone, ethyl acetate, butyl acetate, pentyl acetate, tetrahydrofuran, ethyl ethoxypropionate, N-methyl-pyrrolidone, dimethylacetamide and dimethylformamide solvent naphtha, SOLVESSO 100 or HYDROSOL (ARAL), ethers, or mixtures thereof.

The compositions of the present invention may further include any of a variety of additives such as defoamers, devolatilizers, surfactants, thickeners, flow control additives, colorants (including pigments and dyes) and surface additives.

The compositions of the invention may be contacted with a substrate by any methods known to those skilled in the art, including but not limited to, spraying, dipping, flow coating, rolling, brushing, pouring, and the like. In some embodiments, the inventive compositions may be applied in the form of paints or lacquers onto any compatible substrate, such as, for example, metals, plastics, ceramics, glass, and natural materials. In certain embodiments, the composition is applied as a single layer. In other embodiments, the inventive composition may be applied as multiple layers as needed.

### EXAMPLES

The non-limiting and non-exhaustive examples that follow are intended to further describe various non-limiting and non-exhaustive embodiments without restricting the scope of the embodiments described in this specification. All quantities given in “parts” and “percents” are understood to be by weight, unless otherwise indicated. Although the present invention is described in the instant Examples in the context of a coating, those skilled in the art will appreciate it can also be equally applicable to adhesives, castings, composites, and sealants.

The following Materials were used in Preparing the Compositions of the Examples:
CURING AGENT A a liquid blocked amine (ketimine) for polyisocyanate resins on a cycloaliphatic diamine, from Covestro as DESMOPHEN VP LS 2965;
CURING AGENT B isophorone diamine (IPDA);
CURING AGENT C a low viscosity ketimine curing agent, commercially available from Air Products as ANCAMINE 2458;
CURING AGENT D an aliphatic secondary amine curative for polyurea and polyurethane/polyurea hybrid formulations commercially available from Dorf Ketal as CLEARLINK 1000;
CURING AGENT E a solvent-free latent curing agent for polyisocyanate based on urethane bisoxazolidine, commercially available from Covestro as HARDENER OZ;
CURING AGENT F a low viscosity bis-oxazolidine reactive diluent, commercially available as INCOZOL LV from Industrial Copolymers Ltd.;
CURING AGENT G a liquid blocked amine (aldimine) for polyisocyanate resins on a cycloaliphatic diamine, commercially available from Evonik Industries as VESTAMIN A 139;
CURING AGENT H a liquid blocked amine (aldimine) prepared from triaminononane (TAN), as follows: a 1-liter, 3-neck round bottom flask was equipped with a mechanical stirrer, a heating mantle, a thermocouple, a condenser and a nitrogen purge. 1.20 eq. of the isobutyraldehyde were charged under N₂. 1.00 eq. of the TAN was added to it dropwise so that the temperature remained below 60° C. After the addition was complete, the reaction mixture was heated to 80° C. and held for one hour at this temperature. The stirrer was then turned off to cause separation of the aqueous layer (two to four hours at 80° C.). After separating the aqueous layer, the excess isobutyraldehyde was distilled off first at 110° C. for 30 minutes at standard pressure, then 90 minutes under full vacuum (20 mbar);
CURING AGENT I a liquid blocked (ketimine) prepared from TAN as follows: a 1-liter, 3-neck round bottom flask was equipped with a mechanical stirrer, an oil bath, a thermocouple, a Dean-Stark trap, a condenser and a nitrogen purge. 2 eq. of 4-methyl-2-pentanone were placed under N₂ and stirred at room temperature for 15 min. 1 eq. of amine was then slowly poured into the ketone. The reaction mixture was stirred for 30 minutes without heating. Thereafter, the catalyst, p-toluenesulfonic acid (300 ppm) was added to the mixture. The mixture was then slowly heated to reflux. Water was periodically drained. If the theoretical amount of water could not be reached, the temperature was increased by a further 10′C to initiate more water generation. The reaction mixture was maintained at the elevated temperature for about one hour or up to a constant water level in the Dean-Stark trap. The mixture was then cooled to 50′C and the excess 4-methyl-2-pentanone was removed under vacuum. After completion of the distillation, a full vacuum (20 mbar) was applied for one hour;
CURING AGENT J a liquid blocked amine (aldimine) prepared from JEFFAMINE D 2000 using the same procedure as was used to make CURING AGENT H;
CURING AGENT K a liquid blocked amine (ketimine) prepared from JEFFAMINE D 2000 using the same procedure as was used to make CURING AGENT I;
CURING AGENT L a liquid blocked amine (aldimine) prepared from JEFFAMINE T 403 using the same procedure as was used to make CURING AGENT H;
CURING AGENT M a liquid blocked amine (ketimine) prepared from JEFFAMINE T 403 using the same procedure as was used to make CURING AGENT I;
CURING AGENT N triaminononane (TAN);
CURING AGENT O a polyether amine curative commercially available from Huntsman as JEFFAMINE D 2000;
CURING AGENT P a polyether amine curative commercially available from Huntsman as JEFFAMINE T 403;
ADDITIVE A a surface additive on polyacrylate-basis for solvent-borne coating systems and printing inks, commercially available from BYK Chemie as BYK 358N;
ADDITIVE B a silicone surface additive for solvent-borne coating systems and printing inks, commercially available from BYK Chemie as BYK 306;
URETDIONE A a 1-isocyanato-3-isocyanatomethyl-3,5,5-trimethyl cyclohexane (isophorone diisocyanate or IPDI)-based uretdione, commercially available from Covestro as CRELAN EF 403;
URETDIONE B a polyuretdione prepolymer prepared from DESMODUR XP 2730 (polyisocyanate having uretdione groups, prepared from HDI, available from Covestro LLC, Pittsburgh, Pa.) /2,2,4-trimethyl-1,3-pentanediol (TMPD)/2-ethyl hexanol was prepared using a diol to monol equivalent ratio of 2.8:1 in sufficient amount to substantially consume any free isocyanate groups present. The resin was prepared at 65% solids in BA, having an 889 average uretdione equivalent weight; and
URETDIONE C: a 1-isocyanato-3-isocyanatomethyl-3,5,5-trimethyl cyclohexane (isophorone diisocyanate or IPDI)-based uretdione prepared in Covestro LLC labs that has similar backbone to, but lower functionality than, CRELAN EF 403 (URETDIONE A above).

All formulations in Examples 1 through 38 were prepared following the same procedure. For instance, formulation Example 9 (Table II) was prepared as follows. In a 100 mL plastic container, 2.29 parts of CROSSLINKER G, 0.12 parts ADDITIVE A, 2.30 parts n-butyl acetate were added. The resulting composition was mixed using a FLACKTEK speed mixer for two minutes. Then, 20.29 parts URETDIONE A solution (50% in n-butyl acetate) was added to the mixture. The resulting mixture was mixed for additional two minutes followed by application using a drawdown bar onto test panels.

For evaluation of microhardness and MEK Double Rubs, test samples were prepared by applying 4 mils (100 μm) wet film (2 mils (50 μm) dry) thickness of the formulation onto zinc phosphate treated ACT B952 4″×12″ (10.2 cm×30.5 cm) steel test panels (B952 P60 Cold Roll Steel).

Microhardness (Marten's hardness) measurements were made using a FISCHERSCOPE H100C instrument according to the method described in DIN EN ISO 14577. Microhardness readings were taken under a 20 mN test load run to a maximum of 5 μm indentation depths over a 20 second application time. Results reported were an average of three readings for each formulation.

MEK double rubs were measured using the method described in ASTM D4752-10(2015). Results reported were an average of three readings for each formulation.

Viscosities of the formulations were measured according to ASTM D7395-07(2012) using a BROOKFIELD RST Rheometer at 25° C., 100 s-1 shear rate for two minutes with a RST-50-1 spindle. “Initial viscosity” refers to the viscosity of the composition at the time it was made and “1 hour viscosity” refers to the viscosity of the composition after one hour of being made.

Table I provides the components normalized to 100 by weight combined for each Example along with the results (MEK double rubs and microhardness testing of the materials produced in Examples 1-8). As can be appreciated by reference to Table I, coatings formulated with aldimine and ketimine in Examples 7 and 8 provided good hardness with high MEK double rubs (chemical resistance).

Table II provides the formulation ingredients for similar experiments with pot-life data added to show extension of pot-life along with Marten's Hardness, MEK double rubs. Table III provides viscosity measurements for the materials produced in Examples 9-20. As can be appreciated by reference to Tables II and III, Examples 17 and 19 are non-blocked amines and have a very short pot-life. Examples 9, 10, 13, and 14 are blocked amines which show good hardness, chemical resistance and a long pot-life. The same observation can be seen in comparing Examples 21, 22, and 27 as well as comparing Examples 25, 26, and 29 in Table IV, and Table V. Lastly as can be appreciated from Table VI and Table VII, similar observations are seen by comparing Examples 30, 31, and 36 as well as comparing Examples 34, 35, and 38 within themselves.

The compositions of the present invention are particularly applicable in or as coatings, adhesives, castings, composites, and sealants with good performance and extended pot-life.

TABLE I

| | | | | | | | |
|---|---|---|---|---|---|---|---|
| Ex. 1 | Ex. 2 | Ex. 3 | Ex. 4 | Ex. 5 | Ex. 6 | Ex. 7 | Ex. 8 |

Component 1

| | | | | | | | | |
|---|---|---|---|---|---|---|---|---|
| CURING AGENT A | | | 6.34 | | | | | 10.56 |
| CURING AGENT E | | 4.80 | | | | 8.18 | | |
| CURING AGENT F | 3.63 | | | | 6.30 | | | |
| CURING AGENT G | | | | 5.43 | | | 9.17 | |
| ADDITIVE B | 0.40 | 0.40 | 0.40 | 0.40 | | | | |
| ADDITIVE A | | | | | 0.48 | 0.48 | 0.48 | 0.48 |
| n-butyl acetate (BA) | 24.71 | 25.34 | 26.17 | 25.68 | 6.32 | 8.20 | 9.18 | 10.58 |

Component 2

| | | | | | | | | |
|---|---|---|---|---|---|---|---|---|
| URETDIONEA | | | | | 86.90 | 83.14 | 81.17 | 78.39 |
| (50% in BA) | | | | | | | | |
| URETDIONE B | 71.26 | 69.46 | 67.09 | 68.49 | | | | |
| Total | 100.0 | 100.0 | 100.0 | 100.0 | 100.0 | 100.0 | 100.0 | 100.0 |

Marten's Hardness (N/mm²)

| | | | | | | | | |
|---|---|---|---|---|---|---|---|---|
| 1 Day | | | 1.10 | 0.40 | 2.00 | 1.90 | 4.50 | 20.50 |
| 7 Day | 0.42 | 0.34 | 8.62 | 4.79 | 123.10 | 116.59 | 139.68 | 142.16 |

MEK Double Rubs

| | | | | | | | | |
|---|---|---|---|---|---|---|---|---|
| 1 Day | 5 | 5 | 15 | 5 | 5 | 5 | 10 | 25 |
| 7 Day | 10 | 20 | 30 | 25 | 10 | 5 | 80 | 380 |

TABLE II

| | | | | | | | | | | | |
|---|---|---|---|---|---|---|---|---|---|---|---|
| Ex. 9 | Ex. 10 | Ex. 11 | Ex. 12 | Ex. 13 | Ex. 14 | Ex. 15 | Ex. 16 | Ex. 17 | Ex. 18 | Ex. 19 | Ex. 20 |

Component 1

| | | | | | | | | | | | | |
|---|---|---|---|---|---|---|---|---|---|---|---|---|
| CURING AGENT B | | | | | | | | | 1.50 | | 0.86 | |
| CURING AGENT F | | | | 1.58 | | | | 0.90 | | | | |
| CURING AGENT C | | 1.87 | | | | 1.09 | | | | | | |
| CURING AGENT D | | | | | | | | | | 2.56 | | 1.53 |
| CURING AGENT G | 2.29 | | | | 1.35 | | | | | | | |
| CURING AGENT E | | | 2.05 | | | | 1.20 | | | | | |
| ADDITIVE A | 0.12 | 0.12 | 0.12 | 0.12 | 0.12 | 0.12 | 0.12 | 0.12 | 0.12 | 0.12 | 0.12 | 0.12 |
| n-Butyl acetate | 2.30 | 1.88 | 2.05 | 1.58 | 6.47 | 6.33 | 6.39 | 6.23 | 1.51 | 2.57 | 6.21 | 6.57 |

Component 2

| | | | | | | | | | | | | |
|---|---|---|---|---|---|---|---|---|---|---|---|---|
| URETDIONE A | 20.29 | 21.13 | 20.79 | 21.73 | | | | | 21.87 | 19.75 | | |
| (50% in BA) | | | | | | | | | | | | |
| URETDIONE B | | | | | 17.05 | 17.46 | 17.30 | 17.74 | | | 17.81 | 16.78 |
| Total | 25.0 | 25.0 | 25.0 | 25.0 | 25.0 | 25.0 | 25.0 | 25.0 | 25.0 | 25.0 | 25.0 | 25.0 |

Marten's Hardness (N/mm²)

| | | | | | | | | | | | | |
|---|---|---|---|---|---|---|---|---|---|---|---|---|
| 1 Day | 3.0 | 32.1 | 0.9 | 1.2 | 0.3 | 0.3 | tacky | tacky | 69.1 | 1.5 | 2.4 | tacky |
| 7 Day | 33.2 | 105.1 | 5.1 | 3.7 | 0.2 | 1.0 | 0.3 | tacky | 145.9 | 14.3 | 6.2 | tacky |

MEK Double Rubs

| | | | | | | | | | | | | |
|---|---|---|---|---|---|---|---|---|---|---|---|---|
| 1 Day | 15 | 160 | 10 | 10 | 10 | 95 | 5 | 5 | 20 | 5 | 10 | 5 |
| 7 Day | 5 | 380 | 10 | 10 | 5 | 80 | 5 | 5 | 15 | 25 | 5 | 5 |

TABLE III
Viscosity (cPs)

| Time | Ex. 9 | Ex. 10 | Ex. 11 | Ex. 12 | Ex. 13 | Ex. 14 | Ex. 15 | Ex. 16 | Ex. 17 | Ex. 18 | Ex. 19 | Ex. 20 |
|---|---|---|---|---|---|---|---|---|---|---|---|---|
| Initial | 1064 | 1350 | 1247 | 1324 | 86 | 107 | 120 | 111 | 5692 | 957 | 51 | 101 |
| 1 hour | 1074 | 2752 | 1213 | 1321 | 102 | 112 | 118 | 106 | gel | 1027 | gel | 108 |
| 2 hours | 1127 | 3389 | 1318 | 1281 | 97 | 113 | 118 | 107 | gel | 1017 | gel | 102 |
| 3 hours | 1194 | 3704 | 1233 | 1323 | 96 | 112 | 116 | 110 | gel | 1031 | gel | 100 |
| 4 hours | 1219 | 4256 | 1273 | 1355 | 95 | 116 | 119 | 110 | gel | 1090 | gel | 100 |
| 5 hours | 1302 | 4701 | 1329 | 1373 | 98 | 132 | 137 | 113 | gel | 1231 | gel | 106 |
| 6 hours | 1357 | 5684 | 1304 | 1377 | 103 | 135 | 128 | 113 | gel | 1299 | gel | 107 |
| 7 hours | 1456 | 6210 | 1401 | 1487 | 105 | 145 | 123 | 113 | gel | 1447 | gel | 104 |

TABLE IV

| | | | | | | | | |
|---|---|---|---|---|---|---|---|---|
| Ex. 21 | Ex. 22 | Ex. 23 | Ex. 24 | Ex. 25 | Ex. 26 | Ex. 27 | Ex. 28 | Ex. 29 |

Component 1

| | | | | | | | | | |
|---|---|---|---|---|---|---|---|---|---|
| CURING AGENT H | 3.29 | | | | | | | | |
| CURING AGENT I | | 3.96 | | | | | | | |
| CURING AGENT J | | | 13.09 | | | | | | |
| CURING AGENT K | | | | 13.20 | | | | | |
| CURING AGENT L | | | | | 5.22 | | | | |
| CURING AGENT M | | | | | | 5.74 | | | |
| CURING AGENT N | | | | | | | 1.85 | | |
| CURING AGENT O | | | | | | | | 12.86 | |
| CURING AGENT P | | | | | | | | | 4.11 |
| ADDITIVE A | 0.19 | 0.19 | 0.19 | 0.19 | 0.19 | 0.19 | 0.19 | 0.19 | 0.19 |
| n-Butyl acetate | 10.97 | 11.32 | 16.24 | 16.30 | 12.00 | 12.28 | 10.19 | 16.12 | 11.41 |

Component 2

| | | | | | | | | | |
|---|---|---|---|---|---|---|---|---|---|
| URETDIONE B | 25.55 | 24.53 | 10.48 | 10.30 | 22.59 | 21.79 | 27.77 | 10.83 | 24.29 |
| Total | 40.0 | 40.0 | 40.0 | 40.0 | 40.0 | 40.0 | 40.0 | 40.0 | 40.0 |

Marten's Hardness (N/mm²)

| | | | | | | | | | |
|---|---|---|---|---|---|---|---|---|---|
| 1 Day | 0.27 | 2.71 | Tacky | Tacky | 0.31 | 0.70 | gelled | Tacky | 1.67 |
| 7 Day | 3.13 | 3.66 | Tacky | Tacky | 0.75 | 1.80 | gelled | Tacky | 2.31 |

MEK Double Rubs

| | | | | | | | | | |
|---|---|---|---|---|---|---|---|---|---|
| 1 Day | 10 | 60 | — | — | 5 | 30 | — | — | 35 |
| 7 Day | 35 | 80 | — | — | 5 | 35 | — | — | 60 |

TABLE V

| | Ex. | Ex. | Ex. | Ex. | Ex. | Ex. | Ex. | Ex. | Ex. |
|---|---|---|---|---|---|---|---|---|---|
| Time | 21 | 22 | 23 | 24 | 25 | 26 | 27 | 28 | 29 |

Viscosity (cPs)

| | | | | | | | | | |
|---|---|---|---|---|---|---|---|---|---|
| Initial | 77 | 70 | 18 | 18 | 53 | 51 | gel | 25 | 106 |
| 1 hour | 73 | 94 | 18 | 55 | 53 | 55 | gel | 34 | gel |
| 2 hours | 78 | 97 | 18 | 35 | 53 | 56 | gel | 44 | gel |
| 3 hours | 75 | 110 | 17 | 44 | 63 | 57 | gel | 49 | gel |
| 4 hours | 95 | 137 | 19 | 47 | 62 | 65 | gel | 66 | gel |
| 5 hours | 90 | 157 | 18 | 31 | 61 | 67 | gel | 81 | gel |
| 6 hours | 90 | 177 | 24 | 23 | 62 | 70 | gel | 109 | gel |
| 7 hours | 95 | 216 | 28 | 30 | 65 | 77 | gel | 125 | gel |

TABLE VI

| | | | | | | | | |
|---|---|---|---|---|---|---|---|---|
| Ex. 30 | Ex. 31 | Ex. 32 | Ex. 33 | Ex. 34 | Ex. 35 | Ex. 36 | Ex. 37 | Ex. 38 |

Component 1

| | | | | | | | | | |
|---|---|---|---|---|---|---|---|---|---|
| CURING AGENT H | 2.97 | | | | | | | | |
| CURING AGENT I | | 3.58 | | | | | | | |
| CURING AGENT J | | | 12.53 | | | | | | |
| CURING AGENT K | | | | 12.64 | | | | | |
| CURING AGENT L | | | | | 4.76 | | | | |
| CURING AGENT M | | | | | | 5.25 | | | |
| CURING AGENT N | | | | | | | 1.65 | | |
| CURING AGENT O | | | | | | | | 12.29 | |
| CURING AGENT P | | | | | | | | | 3.72 |
| ADDITIVE A | 0.19 | 0.19 | 0.19 | 0.19 | 0.19 | 0.19 | 0.19 | 0.19 | 0.19 |
| n-Butyl acetate | 2.98 | 3.59 | 12.53 | 12.65 | 4.76 | 5.25 | 1.66 | 12.29 | 3.73 |

Component 2

| | | | | | | | | | |
|---|---|---|---|---|---|---|---|---|---|
| URETDIONE C | 33.86 | 32.64 | 14.75 | 14.51 | 30.29 | 29.31 | 36.50 | 15.23 | 32.36 |
| Total | 40.0 | 40.0 | 40.0 | 40.0 | 40.0 | 40.0 | 40.0 | 40.0 | 40.0 |

Marten's Hardness (N/mm²)

| | | | | | | | | | |
|---|---|---|---|---|---|---|---|---|---|
| 1 Day | 4.53 | 42.22 | Tacky | Tacky | 1.47 | 2.51 | gelled | Tacky | 6.49 |
| 7 Day | 33.18 | 101.69 | Tacky | Tacky | 11.13 | 47.90 | gelled | Tacky | 86.53 |

MEK Double Rubs

| | | | | | | | | | |
|---|---|---|---|---|---|---|---|---|---|
| 1 Day | 5 | 105 | — | — | 10 | 30 | — | — | 40 |
| 7 Day | 20 | 375 | — | — | 10 | 55 | — | — | 55 |

Table VII

| | Ex. | Ex. | Ex. | Ex. | Ex. | Ex. | Ex. | Ex. | Ex. |
|---|---|---|---|---|---|---|---|---|---|
| Time | 30 | 31 | 32 | 33 | 34 | 35 | 36 | 37 | 38 |

Viscosity (cPs)

| | | | | | | | | | |
|---|---|---|---|---|---|---|---|---|---|
| Initial | 142 | 137 | 31 | 36 | 121 | 115 | gel | 61 | 267 |
| 1 hour | 259 | 277 | 52 | 37 | 156 | 148 | gel | 54 | 415 |
| 2 hours | 270 | 523 | 43 | 40 | 160 | 150 | gel | 62 | 370 |
| 3 hours | 290 | 719 | 38 | 41 | 164 | 156 | gel | 67 | 875 |
| 4 hours | 281 | 1307 | 41 | 43 | 172 | 159 | gel | 72 | 1815 |
| 5 hours | 307 | gel | 77 | 36 | 172 | 167 | gel | 70 | 2365 |
| 6 hours | 323 | gel | 46 | 51 | 169 | 180 | gel | 68 | 3062 |

This specification has been written with reference to various non-limiting and non-exhaustive embodiments. However, it will be recognized by persons having ordinary skill in the art that various substitutions, modifications, or combinations of any of the disclosed embodiments (or portions thereof) may be made within the scope of this specification. Thus, it is contemplated and understood that this specification supports additional embodiments not expressly set forth herein. Such embodiments may be obtained, for example, by combining, modifying, or reorganizing any of the disclosed steps, components, elements, features, aspects, characteristics, limitations, and the like, of the various non-limiting embodiments described in this specification. In this manner, Applicants reserve the right to amend the claims during prosecution to add features as variously described in this specification, and such amendments comply with the requirements of 35 U.S.C. § 112(a), and 35 U.S.C. § 132(a).

Various aspects of the subject matter described herein are set out in the following numbered clauses:

1. A composition comprising: a polyuretdione resin; a blocked amine; and optionally, an additive package selected from the group consisting of flow control additives, wetting agents, and solvents.

2. The composition according to clause 1, wherein the polyuretdione resin comprises the reaction product of catalytic dimerization of an isocyanate.

3. The composition according to clause 2, wherein the isocyanate is selected from the group consisting of 1,4-tetramethylene diisocyanate, 1,6-hexamethylene diisocyanate (HDI), 2,2,4-trimethyl-1,6-hexamethylene diisocyanate, 1,12-dodecamethylene diisocyanate, cyclohexane-1,3- and 1,4-diisocyanate, 1-isocyanato-2-isocyanato-methyl cyclopentane, 1-isocyanato-3-isocyanatomethyl-3,5,5-trimethyl cyclohexane (isophorone diisocyanate or IPDI), bis-(4-isocyantocyclohexyl)methane, 1,3- and 1,4-bis-(isocyanatomethyl)-cyclohexane, bis-(4-isocyanato-3-methyl-cyclohexyl)-methane, α,α,α′,α′-tetramethyl-1,3- and/or 1,4-xylene diisocyanate, 1-isocyanato-1-methyl-4(3)-isocyanato-methyl cyclohexane, and 2,4- and/or 2,6-hexahydro-toluene diisocyanate, toluene diisocyanate (TDI), diphenylmethane diisocyanate (MDI), PDI (pentane diisocyanate-bio-based), and, isomers of any of these.

4. The composition according to one of clauses 1 to 3, wherein the blocked amine is selected from the group consisting of aldimines, ketimines, oxazolidines, and combinations thereof.

5. The composition according to clause 1, wherein the blocked amine is an aldimine comprising an aldehyde and an amine.

6. The composition according to clause 5, wherein the aldehyde is selected from the group consisting of acetaldehyde, formaldehyde, propionaldehyde, isobutyraldehyde, n-butyraldehyde, heptaldehyde, cyclohexyl aldehydes, and combinations thereof.

7. The composition according to clause 5, wherein the amine is selected from the group consisting of ethylene diamine, ethylene triamine, propylene diamine, tetramethylene diamine, 1,6-hexamethylene diamine, bis(6-aminohexyl)ether, tricyclodecane diamine, N,N′-imethyldiethyltriamine, cyclohexyl-1,2,4-triamine, cyclohexyl-1,2,4,5-tetraamine, 3,4,5-triaminopyran, 3,4-diaminofuran, and cycloaliphatic diamines, triaminononane, Jeffamines (polyoxyalkyleneamines, polyethers which contain primary amine groups at the end of the polyether backbone), and combinations thereof.

8. The composition according to clause 1, wherein the blocked amine is a ketimine comprising a ketone and an amine.

9. The composition according to clause 8, wherein the ketone is selected from the group consisting of acetone, methyl ethyl ketone, methyl isopropyl ketone, methyl isobutyl ketone, diethyl ketone, benzyl methylketone, diisopropyl ketone, cyclopentanone, cyclohexanone, and combinations thereof.

10. The composition according to clause 8, wherein the amine is selected from the group consisting of ethylene diamine, ethylene triamine, propylene diamine, tetramethylene diamine, 1,6-hexamethylene diamine, bis(6-aminohexyl)ether, tricyclodecane diamine, N,N′-dimethyl-diethyltriamine, cyclohexyl-1,2,4-triamine, cyclohexyl-1,2,4,5-tetraamine, 3,4,5-triaminopyran, 3,4-diaminofuran, and cycloaliphatic diamines, triaminononane, Jeffamines (polyoxyalkyleneamines, polyethers which contain primary amine groups at the end of the polyether backbone), and combinations thereof.

11. The composition according to clause 1, wherein the blocked amine is an oxazolidine selected from the group consisting of 4-propyl oxazolidine, 5-methyl oxazolidine, 2,2-dimethyl oxazolidine, 1-butyl-2,2-dimethyl oxazolidine, 1-methyl-2-butyl oxazolidine, 1-ethyl oxazolidine, 1-ethyl-2,2-dimethyl oxazolidine, 1-ethyl-2-isopropyl oxazolidine, carbonato-bis-N-ethyl-2-isopropyl-1,3-oxazolidine, 2-(3-heptyl)-N-butyl-1,3-oxazolane, bis-oxazolidines, urethane bis-oxazolidines, and combinations thereof.

12. The composition according to clause 1, wherein the solvent is selected from the group consisting of toluene, xylene, isooctane, acetone, butanone, methyl ethyl ketone, methyl amyl ketone, methyl isobutyl ketone, ethyl acetate, butyl acetate, pentyl acetate, tetrahydrofuran, ethyl ethoxypropionate, N-methyl-pyrrolidone, dimethylacetamide, dimethylformamide solvent naphtha, ethers, and mixtures thereof.

13. The composition according to clause 1, wherein the polyuretdione resin contains water.

14. The composition according to clause 1, wherein the blocked amine contains water.

15. One of a coating, an adhesive, a casting, a composite, and a sealant comprising the composition according to clause 1.

16. The composition according to clause 1, wherein the application comprises at least one of spraying, dipping, flow coating, rolling, brushing, and pouring.

17. A method of forming a biuret comprising the steps of: unblocking a blocked amine in the presence of moisture; and crosslinking the unblocked amine with a polyuretdione resin.

18. The method according to clause 17, wherein the polyuretdione resin is made by catalytic dimerization of an isocyanate.

19. The method according to clause 18, wherein the isocyanate is selected from the group consisting of 1,4-tetramethylene diisocyanate, 1,6-hexamethylene diisocyanate (HDI), 2,2,4-trimethyl-1,6-hexamethylene diisocyanate, 1,12-dodecamethylene diisocyanate, cyclohexane-1,3- and 1,4-diisocyanate, 1-isocyanato-2-isocyanato-methyl cyclopentane, 1-isocyanato-3-isocyanatomethyl-3,5,5-trimethyl cyclohexane (isophorone diisocyanate or IPDI), bis-(4-isocyanatocyclohexyl)methane, 1,3- and 1,4-bis(isocyanatomethyl)-cyclohexane, bis-(4-isocyanato-3-methyl-cyclohexyl)-methane, α,α,α′,α′-tetramethyl-1,3- and/or 1,4-xylene diisocyanate, 1-isocyanato-1-methyl-4(3)-isocyanato-methyl cyclohexane, and 2,4- and/or 2,6-hexahydrotoluene diisocyanate, toluene diisocyanate (TDI), diphenylmethane diisocyanate (MDI), pentane diisocyanate (PDI)-bio-based, and, isomers of any of these.

20. The method according to clause 17, wherein the blocked amine is selected from the group consisting of aldimines, ketimines, oxazolidines, and combinations thereof.

21. The method according to clause 17, wherein the blocked amine is an aldimine made by reacting an aldehyde with an amine.

22. The method according to clause 21, wherein the aldehyde is selected from the group consisting of acetaldehyde, formaldehyde, propionaldehyde, isobutyraldehyde, n-butyraldehyde, heptaldehyde, cyclohexyl aldehydes, and combinations thereof.

23. The method according to clause 21, wherein the amine is selected from the group consisting of ethylene diamine, ethylene triamine, propylene diamine, tetramethylene diamine, 1,6-hexamethylene diamine, bis(6-aminohexyl)ether, tricyclodecane diamine, N,N′-dimethyldiethyltriamine, cyclohexyl-1,2,4-triamine, cyclohexyl-1,2,4,5-tetraamine, 3,4,5-triaminopyran, 3,4-diaminofuran, and cycloaliphatic diamines, triaminononane, Jeffamines (polyoxyalkyleneamines, polyethers which contain primary amine groups at the end of the polyether backbone), and combinations thereof.

24. The method according to clause 17, wherein the blocked amine is a ketimine made by reacting a ketone with an amine.

25. The method according to clause 24, wherein the ketone is selected from the group consisting of acetone, methyl ethyl ketone, methyl isopropyl ketone, methyl isobutyl ketone, diethyl ketone, benzyl methylketone, diisopropyl ketone, cyclopentanone, cyclohexanone, and combinations thereof.

26. The method according to clause 24, wherein the amine is selected from the group consisting of ethylene diamine, ethylene triamine, propylene diamine, tetramethylene diamine, 1,6-hexamethylene diamine, bis(6-aminohexyl)ether, tricyclodecane diamine, N,N′-dimethyldiethyltriamine, cyclohexyl-1,2,4-triamine, cyclohexyl-1,2,4,5-tetraamine, 3,4,5-triaminopyran, 3,4-diaminofuran, and cycloaliphatic diamines, triaminononane, Jeffamines (polyoxyalkyleneamines, polyethers which contain primary amine groups at the end of the polyether backbone), and combinations thereof.

27. The method according to clause 17, wherein the blocked amine is an oxazolidine selected from the group consisting of 4-propyl oxazolidine, 5-methyl oxazolidine, 2,2-dimethyl oxazolidine, 1-butyl-2,2-dimethyl oxazolidine, 1-methyl-2-butyl oxazolidine, 1-ethyl oxazolidine, 1-ethyl-2,2-dimethyl oxazolidine, 1-ethyl-2-isopropyl oxazolidine, carbonato-bis-N-ethyl-2-isopropyl-1,3-oxazolidine, 2-(3-heptyl)-N-butyl-1,3-oxazolane, bis-oxazolidines, urethane bis-oxazolidines, and combinations thereof.

28. One of a coating, an adhesive, a casting, a composite, and a sealant comprising the biuret made according to the method of clause 17.

29. A method of applying the biuret made according to the method of clause 17 to a surface, wherein the method comprises at least one of spraying, dipping, flow coating, rolling, brushing, and pouring.

What is claimed is:

1. A composition comprising:
a polyuretdione resin;
a blocked amine; and
optionally,
an additive package and
a solvent.

2. The composition according to claim 1, wherein the polyuretdione resin comprises the reaction product of catalytic dimerization of an isocyanate selected from the group consisting of 1,4-tetramethylene diisocyanate, 1,6-hexamethylene diisocyanate (HDI), 2,2,4-trimethyl-1,6-hexamethylene diisocyanate, 1,12-dodecamethylene diisocyanate, cyclohexane-1,3- and 1,4-diisocyanate, 1-isocyanato-2-isocyanato-methyl cyclopentane, 1-isocyanato-3-isocyanatomethyl-3,5,5-trimethyl cyclohexane (isophorone diisocyanate or IPDI), bis-(4-isocyanatocyclohexyl)methane, 1,3- and 1,4-bis(isocyanatomethyl)-cyclohexane, bis-(4-isocyanato-3-methyl-cyclohexyl)-methane, α,α,α′,α′-tetramethyl-1,3- and/or 1,4-xylene diisocyanate, 1-isocyanato-1-methyl-4(3)-isocyanato-methyl cyclohexane, and 2,4- and/or 2,6-hexahydro-toluene diisocyanate, toluene diisocyanate (TDI), diphenylmethane diisocyanate (MDI), pentane diisocyanate (PDI)-bio-based, and, isomers of any of these.

3. The composition according to claim 1, wherein the blocked amine is selected from the group consisting of aldimines, ketimines, oxazolidines, and combinations thereof.

4. The composition according to claim 1, wherein the blocked amine is an aldimine comprising an aldehyde selected from the group consisting of acetaldehyde, formaldehyde, propionaldehyde, isobutyraldehyde, n-butyraldehyde, heptaldehyde and cyclohexyl aldehydes and an amine selected from the group consisting of ethylene diamine, ethylene triamine, propylene diamine, tetramethylene diamine, 1,6-hexamethylene diamine, bis-(6-aminohexyl)ether, tricyclodecane diamine, N,N′-dimethyl-diethyltriamine, cyclohexyl-1,2,4-triamine, cyclohexyl-1,2,4,5-tetraamine, 3,4,5-triaminopyran, 3,4-diaminofuran, and cycloaliphatic diamines, and combinations thereof.

5. The composition according claim 1, wherein the blocked amine is a ketimine comprising a ketone selected from the group consisting of acetone, methyl ethyl ketone, methyl isopropyl ketone, methyl isobutyl ketone, diethyl ketone, benzyl methylketone, diisopropyl ketone, cyclopentanone, and cyclohexanone and an amine selected from the group consisting of ethylene diamine, ethylene triamine, propylene diamine, tetramethylene diamine, 1,6-hexamethylene diamine, bis-(6-aminohexyl)ether, tricyclodecane diamine, N,N′-dimethyldiethyltriamine, cyclohexyl-1,2,4-triamine, cyclohexyl-1,2,4,5-tetraamine, 3,4,5-triaminopyran, 3,4-diaminofuran, and cycloaliphatic diamines, and combinations thereof.

6. The composition according to claim 1, wherein the blocked amine is an oxazolidine selected from the group consisting of 4-propyl oxazolidine, 5-methyl oxazolidine, 2,2-dimethyl oxazolidine, 1-butyl-2,2-dimethyl oxazolidine, 1-methyl-2-butyl oxazolidine, 1-ethyl oxazolidine, 1-ethyl-2,2-dimethyl oxazolidine, 1-ethyl-2-isopropyl oxazolidine, carbonato-bis-N-ethyl-2-isopropyl-1,3-oxazolidine, 2-(3-heptyl)-N-butyl-1,3-oxazolane, bis-oxazolidines and urethane bis-oxazolidines, and combinations thereof.

7. The composition according to claim 1, wherein the polyuretdione resin contains water.

8. The composition according to claim 1, wherein the blocked amine contains water.

9. One of a coating, an adhesive, a casting, a composite, and a sealant comprising the composition according to claim 1.

10. A method of forming a biuret comprising the steps of:
unblocking a blocked amine in the presence of moisture; and
crosslinking the unblocked amine with a polyuretdione resin.

11. The method according to claim 10, wherein the polyuretdione resin is made by catalytic dimerization of an isocyanate selected from the group consisting of 1,4-tetramethylene diisocyanate, 1,6-hexamethylene diisocyanate (HDI), 2,2,4-trimethyl-1,6-hexamethylene diisocyanate, 1,12-dodecamethylene diisocyanate, cyclohexane-1,3- and 1,4-diisocyanate, 1-isocyanato-2-isocyanato-methyl cyclopentane, 1-isocyanato-3-isocyanatomethyl-3,5,5-trimethyl cyclohexane (isophorone diisocyanate or IPDI), bis-(4-isocyanatocyclohexyl)methane, 1,3- and 1,4-bis(isocyanatomethyl)-cyclohexane, bis-(4-isocyanato-3-methyl-cyclohexyl)-methane, α,α,α′,α′-tetramethyl-1,3- and/or 1,4-xylene diisocyanate, 1-isocyanato-1-methyl-4(3)-isocyanato-methyl cyclohexane, and 2,4- and/or 2,6-hexahydrotoluene diisocyanate, toluene diisocyanate (TDI), diphenylmethane diisocyanate (MDI), pentane diisocyanate (PDI)-bio-based, and, isomers of any of these.

12. The method according to claim 10, wherein the blocked amine is selected from the group consisting of aldimines, ketimines, oxazolidines, and combinations thereof.

13. The method according to claim 10, wherein the blocked amine is an aldimine made by reacting an aldehyde selected from the group consisting of acetaldehyde, formaldehyde, propionaldehyde, isobutyraldehyde, n-butyraldehyde, heptaldehyde and cyclohexyl aldehydes with an amine selected from the group consisting of ethylene diamine, ethylene triamine, propylene diamine, tetramethylene diamine, 1,6-hexamethylene diamine, bis(6-aminohexyl)ether, tricyclodecane diamine, N,N′-dimethyl-diethyltriamine, cyclohexyl-1,2,4-triamine, cyclohexyl-1,2,4,5-tetraamine, 3,4,5-triaminopyran, 3,4-diaminofuran, and cycloaliphatic diamines, and combinations thereof.

14. The method according to claim 10, wherein the blocked amine is a ketimine made by reacting a ketone selected from the group consisting of acetone, methyl ethyl ketone, methyl isopropyl ketone, methyl isobutyl ketone, diethyl ketone, benzyl methylketone, diisopropyl ketone, cyclopentanone, and cyclohexanone with an amine selected from the group consisting of ethylene diamine, ethylene triamine, propylene diamine, tetramethylene diamine, 1,6-hexamethylene diamine, bis-(6-aminohexyl)ether, tricyclodecane diamine, N,N′-dimethyldiethyltriamine, cyclohexyl-1,2,4-triamine, cyclohexyl-1,2,4,5-tetraamine, 3,4,5-triaminopyran, 3,4-diaminofuran, and cycloaliphatic diamines, and combinations thereof.

15. The method according to claim 10, wherein the blocked amine is an oxazolidine selected from the group consisting of 4-propyl oxazolidine, 5-methyl oxazolidine, 2,2-dimethyl oxazolidine, 1-butyl-2,2-dimethyl oxazolidine, 1-methyl-2-butyl oxazolidine, 1-ethyl oxazolidine, 1-ethyl-2,2-dimethyl oxazolidine, 1-ethyl-2-isopropyl oxazolidine, carbonato-bis-N-ethyl-2-isopropyl-1,3-oxazolidine, 2-(3-heptyl)-N-butyl-1,3-oxazolane, bis-oxazolidines and urethane bis-oxazolidines, and combinations thereof.

16. One of a coating, an adhesive, a casting, a composite, and a sealant comprising the biuret made according to the method of claim 10.
